(19) United States
(12) Patent Application Publication
(10) Pub. No.: US 2012/0101172 A1
(43) Pub. Date: Apr. 26, 2012

(54) LIQUID OR POURABLE PROTEOSOME-FORMING BATH AND SHOWER CONCENTRATES, GALENIC APPLICATION PRODUCTS THEREOF, AND THEIR USE

(75) Inventors: Rolf Beutler, Hoechst (DE); Karl Heinz Schmidt, Gomaringen (DE)

(73) Assignee: MERZ PHARMA GMBH & CO. KGAA, Frankfurt/Main (DE)

(21) Appl. No.: 13/380,936

(22) PCT Filed: Jul. 7, 2010
(86) PCT No.: PCT/EP2010/004142

§ 371 Date: Dec. 27, 2011

(30) Foreign Application Priority Data
Jul. 15, 2009 (DE) 10 2009 033 109.3

(51) Int. Cl. A61K 8/92; A61P 29/00; A61P 25/00; A61P 19/00; A61K 47/42

(52) U.S. Cl. 514/773

(57) ABSTRACT

The invention relates to balneological preparations, especially bath and shower preparations, which, as a pourable or liquid premixture concentrate, contain liposome-forming components, such as phospholipids, in addition to natural and/or synthetic lipids, and are also provided as mixtures containing primarily vegetable proteins, surfactants, and optional additives. When entering in contact with an excess of water, the stable systems directly form skin-active proteoliposomes having a catalytic activity for enhancing transfer of active ingredients into the skin. The stable systems can be used directly or in diluted or solid forms of application (such as lipid-containing proteasome shower preparation, proteasome bath powder, and the like).

### OBJECT OF THE INVENTION

The present invention relates to balneological preparations, particularly bath and shower preparations, which, as pourable or liquid premix concentrates, contain not only natural and/or synthetic lipids, but also liposome-forming components such as phospholipids, and furthermore are present as mixtures with predominantly vegetable proteins, surfactants, and, if necessary, additives. These stable systems directly form skin-active proteoliposomes upon contact with an excess of water, with catalytic activity for improved transport of active substance into the skin. They can be used directly or in dilute or solid application forms (such as proteosome shower preparations that contain lipids, proteosome bath powders, and the like).

### STATE OF THE ART

Balneological preparations such as soaps, oils, etc. have been well known to persons skilled in the art, in many varied compositions and forms of administration, since ancient times and the Middle Ages, as components of a spa culture that has been highly developed in many countries. However, basic scientific knowledge and systematic studies concerning specific effects of balneological preparations, such as, for example, cleaning or care or treatment of the skin, have only become available in the past few decades. In contrast to cosmetic preparations such as creams or lotions, for example, which can be applied to the skin in concentrated form and remain there over extended periods of time, balneological preparations are used in greatly diluted form, because of the bath or shower water, and the contact time on the skin is comparatively short during the bath or shower procedure. A person skilled in the art takes this fact into account by distinguishing between cosmetic “leave-on” preparations and balneological “rinse-off” preparations. In order to take this dilution effect into account, the balneological preparations are mostly offered in the form of concentrates, in the state of the art.

Premix concentrates represent a particular form of balneological preparations; these are structured in such a manner that the active balneological system is only formed after they are introduced into an excess of water. DE 42 05 548 A1, for example, describes specific stabilizing, water-dispersible oil-soluble polyoxyethylated surfactants having an HLB value of 6 to 13, together with vesicle-forming agents such as phospholipids, for example, in an oil base, as such a balneological preparation, which forms bath liposomes after introduction into an excess of water, which liposomes have an advantageous skin-active balneological effect.

A person skilled in the art is also familiar with lipoproteins that are able to bind and transport lipids. In DE 103 24 256 A1, for example, proteins are disclosed as vehicles for lipids, which bring about effective lipid transport in catalytic manner. In this connection, energy barriers of the lipid exchange are lowered, and the transfer of lipophilic active substances into aqueous media is made possible or increased.

### CRITICISM OF THE STATE OF THE ART

In the state of the art, no balneological preparations are known that contain the advantageous properties of liposomal systems from premixes, on the one hand, and offer the advantage of catalytic active substance transport by means of lipoproteins, on the other hand.

### TASK OF THE INVENTION

It is therefore the task of the invention to make available stable balneological preparations, particularly bath and shower preparations, which develop skin-active liposomal systems (nano-proteosomes) from premixes that contain protein(s), after contact with an excess of water, and allow additional catalytic active substance transport.

### SOLUTION FOR THE TASK

This task is accomplished by the balneological basic premix concentrates for forming proteo(lipo)somes having lipophilic components, comprising 25 to 95 weight percent of lipids, selected from among vegetable, animal, or synthetic lipids or mixtures thereof, 0.1 to 15 weight percent of one or more substances that form liposomes, selected from among phospholipids, sphingolipids, or mixtures thereof, 0.5 to 70 weight percent of one or more surfactants, such as, for example, ethoxylated fatty alcohols, ethoxylated fatty acids, ethoxylated sugar esters or sugar ethers of fatty acids or fatty alcohols, fatty alcohol sulfates or their ethoxylated derivatives, 0.05 to 15 weight percent of one or more proteins selected from among vegetable proteins having an average molecular mass of 4 kD to 30 kD, milk proteins having an average molecular mass of 2.5 kD to 18 kD, quaternized milk protein having an average molecular mass of 2.5 kD to 12 kD, and, if applicable, additional additives, selected from among active substances, conditioners, pigments, consistency regulators, preservatives. These concentrates preferably comprise less than 5 wt.-%, particularly less than 3%, and above all less than 1 wt.-% water.

With such basic concentrates made from the premix concentrate as described (composed of lipid, liposome-forming agent, protein, + if applicable active substance) and surfactant and, if applicable, additive, liposomal structures that contain protein (nano-proteo(lipo)somes) having lipophilic components are obtained when used with or in an excess (such as, for example, more than six times the amount of water, such as in bathwater or while showering), which structures have the catalytic transport property for lipophilic/amphiphilic active substances, brought about by the proteins, in addition to the liposomal vehicle effect. This is surprising, because on the one hand, lipids are needed in this system, while on the other hand, surfactants can be viewed as disadvantageous with regard to the liposomal effect. Surprisingly, however, an equilibrium can be reached at a ratio of the liposome-forming component(s) such as phospholipids, for example, to the protein component(s) of 10 to 1 to 1 to 1, above all of 3 to 1 to 1 to 1, so that on the one hand, the liposomal vehicle structure, and, on the other hand, the protein-catalyzed active substance transport can be utilized. Surprisingly, it is possible, even at high surfactant contents in the premix/base (basic concentrate) of the balneological preparations according to the invention, to obtain the proteoliposomal structures after dilution in the bathwater or during the shower process. This promotes increased active substance transport into the skin even in the case of the “rinse-off” applications, as is the rule for cosmetic “leave-on” applications.

### FIGURES

FIGS. 1a, b, and 2a, b show the improvement in the epidermal oxygen supply and the cutaneous microcirculation after bathing or showering with balneological preparations according to the invention, having a corresponding basic formulation as described in Example 1 or 4.

FIG. 3 shows an electron-microscope image of the proteoliposomal structures formed after introduction of a balneological preparation according to the invention, in accordance with Example 2, into an excess of water.

### EXPLANATION OF EMBODIMENTS ACCORDING TO THE INVENTION

### Lipids:

The balneological preparations according to the invention contain one or more lipids as a first active ingredient; these are selected, in particular, from vegetable oils or mixtures of vegetable oils with glycerides or fatty acid esters, fatty alcohols, fatty acids, or, if applicable, also with hydrocarbons such as paraffins. These include, above all, olive oil, sunflower oil, soy oil, canola oil, peach pit oil, apricot pit oil, grape seed oil, castor oil, peanut oil, almond oil, wheat germ oil, sesame oil, thistle oil, avocado oil, shea butter, illipé butter, coconut oil, borage oil, maize germ oil, walnut oil, palm oil, macadamia nut oil, palm kernel oil, hazelnut oil, rosehip oil, cottonseed oil, papaya oil, rosewood oil, rosemary oil, eucalyptus oil, thyme oil, pine needle oil, cardamom oil, turpentine oil, jojoba oil or its substitute oleyl erucate.

Particularly preferred vegetable oils are soy oil, sunflower oil, wheat germ oil, almond oil, olive oil or mixtures thereof.

### Liposome-Forming Component:

Phospholipids, for example from egg, soy, rapeseed, cottonseed, such as phosphatidylcholine from soy or egg yolk, mixtures with phosphatidylcholine in different ratios such as NAT products, phosphatidylethanolamine, phosphatidylserine, phosphatidylinositol from soy, egg yolk, rapeseed, cottonseed, furthermore lecithins from soy, egg yolk, rapeseed, cottonseed are particularly suitable as liposome-forming components.

Other suitable components for forming liposomes or vesicular structures are sphingolipids such as ceramides, cerebrosides, sphingomyelins, etc.

The total proportion of liposome-forming components amounts to, above all, 0.2 to 10 weight percent, particularly 0.4 to 5 weight percent, preferably 1 to 3 weight percent.

### Proteins:

According to the invention, vegetable proteins such as those from grains, shellfish, milk are particularly used as proteins or, depending on the molecular mass, protein hydrolysates. Grains that are preferred are wheat, oat, soy, buckwheat, tapioca, maize, barley, rice, beans, millet, spelt, or mixtures thereof. The proteins or hydrolysates have an average molecular mass, above all, from 4,000 to 30,000 D, preferably >10,000 D to 30,000 D, above all 11,000 D to 25,000 D. In particular embodiments, the average molecular mass of the vegetable proteins, particularly grain proteins, for example oat, wheat, barley, soy, can lie between 4,000 and 9,900 D, in other embodiments between >10,000 D to 25,000 D, preferably up to 19,000 D. Proteins or hydrolysates from buckwheat, tapioca, beans, rice proteins having an average molecular mass of 11,000 to 25,000 D, above all up to 11,000 D to 19,000 D, are particularly preferred. Furthermore, rice, bean or also maize, millet, soy-wheat-oat protein, or mixtures thereof, are particularly advantageous, or also rice, bean or maize, millet, soy-wheat-oat protein having an average molecular mass of 12,500 to 18,000 D, particularly also mixtures of soy and oat protein, soy and maize protein, barley and millet protein, in a ratio of 1:1 to 1:5.

Suitable shellfish are those having an average molecular mass of 25,000 to 45,000 D, above all 41,000 D to 45,000 D.

Furthermore, animal proteins or protein hydrolysates are suitable, such as those from milk, having an average molecular mass of 2,500 D to 18,000 D, above all 2,500 to 8,500 D, or also 2,500 to 3,500 D, or, in other embodiments, also above all 9,000 to 15,000 D, particularly 9,000 to 12,000 D. Also suitable are quaternized milk proteins or hydrolysates of them, such as cocodimonium hydroxypropyl milk protein.

Preferred proteins are vegetable proteins, particularly selected from among the grain proteins listed above, or mixtures thereof.

The proteins or hydrolysates to be used according to the invention can be obtained from the starting materials by means of being slurried up in water, if applicable by reaction with acids or bases, further processing such as centrifugation, or applicable removal of accompanying substances by means of precipitation, for example with acid, salts (such as ammonium sulfate), defatting using organic solvents, purification by means of dialysis, gel filtration, chromatography, etc. Methods for this are known in the state of the art and are described, for example, in DE 38 15 473 C1, see also U.S. Pat. No. 5,776,470, U.S. Pat. No. 6,077,529.

The amount of protein(s) preferably amounts to 0.2 to 15 wt.-%, particularly 0.2 to 4 wt.-%, above all 0.3 to 1.1 wt.-%.

Particularly preferred proteins or protein hydrolysates can be selected from among vegetable proteins (average molecular mass 4,500 to 9,000 D, for example, or also particularly 11,000 to 23,000 D), above all from oat, soy, buckwheat, rice, millet; or mixtures thereof; milk protein (average molecular mass 2,500 to 3,500, particularly 9,500 to 15,000 D), quaternized milk protein or mixtures thereof, as well as mixtures of vegetable proteins or mixtures of vegetable and milk proteins as mentioned, such as: buckwheat/oat; oat/millet; rice/milk; buckwheat/milk; oat/milk, oat/quaternized milk protein having the molecular masses indicated, in each instance, particularly the particularly preferred molecular masses.

### Surfactants:

Surfactants that are selected are particularly sugar esters or sugar ethers of glucose, saccharose, methyl glucose of C₁₂-C₁₈ saturated, unsaturated, partially saturated fatty acids; or of polyoxyethylated and/or polyoxypropylated medium-chain and higher-chain (C₁₂-C₂₂) fatty acids (esters); or of C₈-C₂₀ saturated, unsaturated, partially saturated or such polyoxyethylated and/or polyoxypropylated fatty alcohols (ethers). In the case of polyoxyethylated or polyoxypropylated products, the EO (PEG products) or PO (PPG products) can lie between 8 and 40, preferably 8-25. Also, mixed polyoxylated products can be used.

Glucose, saccharose, methyl glucose are particularly suitable as sugars.

Above all, organic aliphatic C₁₂-C₁₈ carboxylic acids or such alcohols are suitable as fatty acids or fatty alcohols. These include, in particular, caprylic acid, caprinic acid, laurinic acid, myristinic acid, palmitinic acid, stearic acid, oleic acid or such alcohols, such as lauryl alcohol, stearyl alcohol, and also mixtures thereof suitable.

Known oxylated (ethoxylated, propoxylated, mixed-oxylated) sugar surfactants are, for example, PEG-20 methyl glucose sesquistearate, PEG-120 methyl glucose dioleate or methyl-gluceth-10, methyl-glueceth-20, PPG-10 methyl glucose ether, PPG-20 methyl glucose ether.

Also, saccharose/palmitate stearate, methyl glucose stearate, decyl glucoside, and mixtures thereof are particularly suitable. Alternatively or in addition, nonionic surfactants on the basis of fatty acids or fatty alcohols, such as nonionic ethoxylated and/or propoxylated fatty alcohols (fatty alcohol ethers), nonionic ethoxylated and/or propoxylated fatty acids (esters) or their amides, such as, for example, mono or diethanolamides, can be used, whereby the fatty alcohol or the fatty acid has a chain length C₈ to C₂₀, or C₁₂ to C₂₂, above all C₁₂ to C₁₈. These include, above all, C₁-C₈-alkyl-C₁₀-C₁₈-fatty alcohol ethoxylates such as polyoxyethylene lauryl ether (1 to 4 EO, laureth 1-4), polyoxyethylene (5) oleyl ether, polyoxyethylene (7) palmityl ether, polyoxyethylene (15) stearyl ether. Ethoxylated products are preferred.

Furthermore, amides of the fatty acids, or ethoxylated fatty acids having a chain length C₈ to C₂₀, above all C₁₂ to C₁₈, such as ethanolamides or diethanolamides, such as coconut fatty acid diethanolamide, are suitable.

Such surfactants preferably have an HLB value of 6 to 16; preferably 7-13 (alone or in a mixture).

Alternatively, anionic fatty alcohol surfactant derivatives can also be used, particularly in combination with nonionic surfactants, such as the aforementioned fatty acid or fatty alcohol derivatives (ethoxylated or propoxylated, preferably ethoxylated derivatives) or nonionic fatty acid amides. The suitable anionic surfactant fatty alcohol derivatives include, above all, C₈ to C₂₀, above all C₁₀ to C₁₈ fatty alcohol sulfates or sulfate salts such as amine salts, particularly monoisopropanolamine sulfate salts, if applicable ethoxylated derivatives of them, with an EO degree of 0 to 4, above all 1-4. These include, for example, MIPA laureth sulfate (monoisopropanolamine (EO1-4) lauryl sulfate.

Surfactants selected from among ethoxylated C₁₀ to C₂₀ fatty alcohols, ethoxylated C₁₂ to C₂₀ fatty acids having an EO or PO degree of 1 to 5, in each instance, above all 1 to 4 amounts to; or C₁₂ to C₂₀ fatty acid amides, particularly mono or diethanolamides, or mixtures thereof, or mixtures thereof with anionic fatty alcohol sulfate salts, for example in a ratio of 3:1 to 1:1 (nonionic:anionic), are particularly preferred. In this connection, the nonionic surfactants can be mixtures of the aforementioned fatty alcohol or fatty acid derivatives (particularly the aforementioned ethoxylated derivatives) and of the aforementioned fatty acid amides, particularly ethoxylated products, for example in a ratio of 2:1 to 1:2.

It is also preferred if at least one surfactant selected from the group comprising ethoxylated and/or propoxylated C₁₀ to C₂₀ fatty alcohols, ethoxylated and/or propoxylated C₁₂ to C₂₀ fatty acids having an EP or PO or EC-PO degree of 1-5, C₁₀ to C₂₀ (if applicable ethoxylated) fatty alcohol (ether) sulfate salts/sulfate monoisopropanolamines having an EO degree of 0.4; or mixtures thereof is present as a surfactant.

The surfactants can be contained in amounts of preferably 4 to 65%, above all 8 to 65 wt.-%, above all 10-65 wt.-%. Depending on the purpose of use, it can be advantageous to use a higher or a lower amount of surfactant, such as, for example, 8-20 wt.-%, in order to achieve a proteoliposomal bath oil effect, or, for example, 40 to 65 wt.-%, particularly 57 to 63 wt.-%, to achieve a proteoliposomal bath foam effect. Neither the high content of lipid, on the one hand, nor the high surfactant content, on the other hand, which is then present in the surrounding bathwater, in each instance, surprisingly prevents the desired formation of proteoliposomal structures, as is evident from the attached figures.

### Additives:

As additives (in total, for example, in an amount of 0 to 30%, above all 0.01-30%, above all 0.01-20%, above all also 0.1 to 15 wt.-% or 1 to 10 wt.-%, active substances that are usually used for cosmetic or, if applicable, medicinal bath additives are suitable, such as, in particular, ether oils/extracts preferably selected from among khaki leaves, mango, figs, lavender oil, cedar wood, lotus blossoms, chamomile blossoms, ylang ylang, gingko, pine needle, cypress, birch leaf extract, aloe vera extract, marigold extract, hibiscus extract, burdock root extract, hamamelis extract, pennywort extract, algae extract, persimmon extract, water lily extract, cinnamon extract, extracts from thyme, mint, limes, oranges, grapefruit, tangerine, juniper, catnip, lemon balm, eucalyptus, thyme, palmarosa, rosemary, lavender, rosewood, lemon grass, wild rose, fir needle, pine needle, ginger extract, currant extract, linden blossom extract, marigold extract, magnolia extract, pineapple extract, guava extract, Echinacea extract, ivy leaf extract, or mixtures thereof.

These extracts are produced in known manner, such as by means of steam distillation. In this way, ether oils, for example, are obtained from the stated plants, which are particularly preferred. The extracts can also be obtained by means of solvent extraction (using alcohols, triglycerides, or hydrocarbons, water, mixtures thereof), and then be used as such. Analogous synthetically produced substances are, for example, terpines and terpenoids such as camphor, menthol, cineol, or mixtures thereof.

Other suitable active substances can be, for example, vitamins such as Vitamin A, E, or other or suitable derivatives thereof such as esters, for example palmitate, acetate, or phosphate.

Other active substances are, for example, antimycotic substances and those that promote circulation and inhibit skin inflammation. These include, for example, clotrimazole, ciclopirox olamine or ketoconazole, antiseptics, antibiotics, for example gentamicin, hormones or corticoids, for example betamethasone as well as its esters (propionate, acetate, valerate), hydrocortisone, methyl prednisolone and its derivatives, triamcinolone acetamide, benzalkonium chloride, chlorhexidine, dexpanthenol; allantoin, bisabolol, astringent and sebum-regulating substances such as Acnacidol 101 (propylene glycol, hydroxydecanoic acid), pyridoxines, niacin amides, methyl glycine, saccharomyces cerevisiae extract be incorporated.

Furthermore, substances that promote circulation can be used as active substances, for example nicotinic acid derivatives such as methyl or tocopheryl nicotinate, alpha and beta hydroxy acids and their derivatives, for example glycolic acid, maleic acid, citric acid, tartaric acid, lactic acid, salicylic acid, isopropyl benzyl salicylates, C12-13 alkyl lactate (Cosmacol® ELI), or also antiphlogistic and antibacterial substances such at triterpenes, for example ursolic acid, glycyrrhinic acid, or glycyrrhetinic acid and their derivatives, for example stearyl glycyrrhetinate, potassium glycyrrhinate; pantothenic acid derivatives, for example D-panthenol, panthenyl triacetate, or also polyphenols, flavonoids, for example rutin, ferulic acid and its esters, or isoflavones such as soy isoflavone or coenzyme Q10.

Preferred active substances are selected from among ether oils, plant extracts, vitamins, or mixtures thereof, particularly also mixtures thereof with glycerin or solvents such as ethanol.

Other suitable additives can be selected from among conditioning agents, preservatives, pigments, perfumes, conditioners, or mixtures thereof. These can particularly be present in amounts of 0.01 to 8%, preferably 0.1 to 5%, in each instance.

Usual conditioning agents in suitable amounts include, for example, acrylamides (polyacrylamide, or mixtures containing polyacrylamide), starch, such as hypromellose or starch glycola, for example, rice starch, wheat starch, maize starch, and potato starch, furthermore also hydrophobically modified starches, and the like. Furthermore, polysaccharides (for example xanthan gum) or silicates such as magnesium aluminum silicate can be mentioned here. Furthermore, complex-forming agents such as EDTA Na salt, means for pH adjustment, for example citric acid, caustic lye, solvents such as propylene glycol, ethanol, glycerol, polyethylene glycol, such as PEG 400 and the like, for example, salts such as NaCl, or mixtures thereof can also be contained as conditioning agents. Further conditioning agents are, in particular, mono and dialkyl phosphates or glyceryl stearate, possibly colloids.

Particularly suitable conditioners are moisturizers such as glycerin, propylene glycol or polyethylene glycols, polypropylene glycol, butylene glycol, sorbitol, or polymers, for example polyquarternium types such as polyquarternium-39, collagen or its hydrolysates, amino acids, urea, polysaccharide, or commercially available moisturizers such as inositol, sodium lactate, DL-2-pyrrolidone-5-carboxylic acid, Na salt.

Pigments and preservatives for such products are known to a person skilled in the art and are particularly selected from among water-soluble products.

Ether oils as mentioned above or known analogous/synthetic substances particularly serve as perfumes.

### Galenic Formulation Agents:

The premix bases described above can be used as such (premix concentrates such as bath oils, shower concentrates having a lipid component or high lipid component, also as indication products such as sports shower concentrate, relaxation bath oil, refreshing shower/bath, and the like). Alternatively, solid bath products such as, for example, bath tablets, bath beads can be prepared from the premix concentrates, using suitable formulation agents. Dilute products such as bath creams, bath foam creams, shower creams or shower foams are obtained by means of reaction with suitable solvents, particularly water. In particular, galenic preparations can be obtained from the concentrates described above, for example in an amount of 5-84% of a concentrate and 95 to 16 wt.-% of one or more galenic aids, selected from among solid regenerative substances, tablet-forming substances, solid gas/foam generators, salt crystals, organic pellets, or liquid solvents, particularly water. A listing of possible products is indicated in Tables 2 to 4 below.

In this connection, suitable galenic formulation agents are, for example, salts such as sodium chloride, sodium carbonate/bicarbonate, calcium or magnesium carbonate/bicarbonate (so-called CO₂ generators for effervescent tablets or powders), or colloids, above all organic colloids, for beads; furthermore also tablet-forming agents, splitting agents, colloidal aids for solid forms. Such aids are known to a person skilled in the art. Solvents are particularly selected from among water or water in a mixture with compatible organic solvents.

Thus, the following embodiments are obtained (see also Table 2 to 4):

Lipid-rich bath creams or bath foams that contain lipids:

16 to 34% water as a galenic aid, as well as 66 to 84% proteosome premix concentrate, comprising:

14-65%; above all 25-67% (lipid-rich) or 5-25% (containing lipid) lipids; 0.2 to 11% transfer protein proteins; 0.2 to 11% liposome-forming substance; 15 to 68%, above all 15 to 34% (lipid-rich) or 25 to 65% (containing lipid) surfactants; remainder additives, if present.

For shower baths, the following preferred embodiments are obtained:

40 to 58% water as a galenic aid, as well as 42 to 60% proteosome premix concentrate, comprising:

8-35% lipid (shower cream), 25-62% lipid (lipid-rich shower foam), 5-25% lipids (shower foam containing lipids); 0.2 to 8% transfer protein proteins; 0.2 to 8% liposome-forming substance; 15 to 59% surfactants; remainder additives, if present (above all active substances).

### Solid Products:

80 to 95% solids (for example NaCl, Na—Mg/CaCO₂ as salt media for powder or tablets, or 90 to 95% colloids (organic pellets), and, if applicable, 0.1 to 8% tablet-forming aids as mentioned above, together with 5 to 20% proteosome premix concentrate.

### Preferred Embodiments

Preferred concentrate preparations comprise the following combinations:

Balneological preparations, comprising 25 to 95% lipids, above all 25 to 50 wt.-%, particularly 25 to 30 wt.-%, or also 70 to 90 wt.-% or selected from among vegetable lipids, C₁₂₋₂₂ fatty acid glycerides, C₂₋₆-alcohol C₁₂₋₂₂ fatty acid esters, C₈₋₂₂ fatty alcohols, or mixtures thereof; 0.1 to 5 wt.-% of one or more liposome-forming substances, selected from among phospholipids, above all lecithins, and sphingolipids, above all sphingosine; or mixtures thereof; 5 to 65 wt.-%, above all 5 to 65 wt.-% or also 57 to 65 wt, or 5 to 15 wt.-% (in correlation with the aforementioned corresponding lipid content, in each instance) of one or more surfactants, selected from among polyoxyethylated and/or polyoxypropylated C₈-C₂₀ fatty alcohols, polyoxyethylated and/or polyoxypropylated C₁₂-C₂₂ fatty acids having an EO/PO degree of 1-4, if applicable ethoxylated C₈-C₂₂ fatty acid amides; anionic, if applicable ethoxylated C₈-C₂₀ fatty acid alcohol sulfate salts, or mixtures thereof; 0.5 to 3 wt.-% of one or more proteins, from plants, having an average molecular mass of >10,000 D to 30,000 D, or milk protein, having an average molecular mass of 2,500 to 18,000 D, 2,500 D to 18,000 D; quaternized milk protein having an average molecular mass or mixtures thereof; and, above all, 0.1 to 20 wt.-% additives, selected from among active substances, conditioners, consistency regulators, preservatives, perfumes, pigments, or mixtures thereof.

In particular, the amount of water amounts to less than 5 wt.-%, particularly less than 1 wt.-%.

In another embodiment of the invention, protein(s) that is/are present is/are grain protein(s) selected from among rice, bean, or also maize, millet, soy, wheat, oat protein or hydrolysates, having an average molecular mass of 11,000-19,000 D, or mixtures thereof; or milk protein having an average molecular mass of 8,500 to 15,000 D; or a mixture of the said milk protein with one or more of the said grain protein(s), for example in a total amount of 0.1 to 3 wt.-%, preferably 0.5 to 1.5 wt.-%.

Preparations are also suitable in which one or more vegetable proteins are contained, particularly one or more grain proteins (for example rice, bean, or also maize, millet, soy, wheat, oat protein or hydrolysates) having an average molecular mass of 11,000 D to 25,000 D, in a mixture with quaternized milk protein having an average molecular mass of 2,500, above all 4,000 to 12,000 D, for example in a total amount of 0.1 to 3 wt.-%, preferably 0.5 to 1.5 wt.-%.

Another advantageous embodiment comprises preparations in which one or more vegetable proteins are present as the protein(s), particularly rice, bean, or also maize, millet, soy, wheat, oat protein or hydrolysates having an average molecular mass of 4,000 to 9,500 D, or mixtures thereof, or milk protein or quaternized milk protein having an average molecular mass of 2,500 to 3,500 or 8,500 to 15,000 D, or mixtures of the said milk protein or quaternized milk protein with one or more of the said wheat proteins.

Furthermore, such preparations in which vegetable oils or vegetable oils in a mixture with C₁₂₋₂₂ fatty acid glycerides and/or C₈₋₂₂ fatty alcohol are contained as the lipids are preferred.

In preparations according to the invention, above all, jojoba oil, almond oil, soy oil, sunflower oil, olive oil, or mixtures thereof or with caprylic-capric triglyceride, glyceryl stearate, are suitable as vegetable lipids.

Preparations that have a rice-bean or also maize, millet, soy-wheat-oat protein or hydrolysates of them, having an average molecular mass of 11,000 D to 19,000 D, or mixtures thereof, or with milk protein having an average molecular mass of 2,500 to 18,000 D, particularly 9,000 to 15,000 D, are particularly advantageous.

Preferably, the protein or the proteins here is/are present in a (total) amount of 0.5 to 3, above all 1.5 wt.-%.

Another embodiment of the invention relates to preparations in which the surfactant or the surfactants is/are selected from among sugar esters of polyoxyethylated and/or polyoxypropylated (C₈-C₁₈) fatty acids (esters); sugar ethers of polyoxyethylated and/or polyoxypropylated C₈-C₂₀ fatty alcohols having a PO or EO degree 3 and 16, particularly 4-8.

Particularly preferred are ethoxylated C₁₀ to C₁₈ fatty alcohols, ethoxylated and/or propoxylated C₁₂ to C₁₈ fatty acids, having an EO or PO degree, in each instance, of 1 to 5, above all 2 to 4; C₁₂ to C₁₈ fatty acid amides, or mixtures thereof.

In another preferred embodiment, anionic, if applicable ethoxylated C₈ to C₂₀ fatty alcohol sulfate salts, such as amine salts, above all substituted amine salts such as mono/diethanolamine, above all monoisopropylamine salts; particularly in combination with nonionic ethoxylated C₈ to C₂₀ fatty alcohol ethers, (ethoxylated) or C₁₂ to C₂₀ fatty acid amides, or mixtures of the last two, are used.

It is furthermore advantageous if the weight ratio of liposome-forming substance(s) to protein(s) amounts to 1:1 to 5:1, particularly 2.5:1 to 1.3:1.

In another embodiment of the invention, 0.1 to 20, above all 18 wt.-%, particularly 0.1 to 10 wt.-% additives, selected from among active substances, consistency regulators, conditioners, preservatives, pigments, perfumes, or mixtures thereof are contained. Particularly preferred active substances are ether oils, plant extracts, vitamins, or mixtures thereof.

In another embodiment, the balneological preparations according to the invention comprise 75 to 90 wt.-% lipids, selected from among vegetable lipids, C₁₂₋₂₂ fatty acid glycerides, C₂₋₆-alcohol C₁₂₋₂₂ fatty acid esters, C₈₋₂₂ fatty alcohols, or mixtures thereof, above all selected from among jojoba oil, caprylic-capric triglyceride, glyceryl stearate, soy oil, sunflower linolenic acid, olive oil, or mixtures thereof, furthermore 5-10 wt.-% surfactants, selected from among ethoxylated and/or propoxylated C₈ to C₂₀ fatty alcohols or sulfate salts thereof, ethoxylated and/or propoxylated C₁₂ to C₂₂ fatty acids, having an EO or PO or EO-PO degree of 1 to 5, in each instance, above all 1 to 4; or mixtures thereof, C₁₂ to C₂₂ fatty acid amides, or mixtures thereof; 2 to 4 wt. of one or more phospholipids, particularly lecithins such as egg, soy, cottonseed, rapeseed lecithin; sphingolipids; or mixtures thereof; 0.5 to 4 wt.-% of one or more proteins or protein hydrolysates, selected from among rice, bean, or also maize, millet, soy, wheat, oat protein having an average molecular mass of 11,000 to 19,000 D, in each instance, milk protein having an average molecular mass of 2,500 D to 18,000 D; quaternized milk protein having an average molecular mass as indicated, as well as 1 to 10 wt.-% additives, selected from among ether oils, plant extracts, vitamins, conditioners, pigments, consistency regulators, preservatives.

In this embodiment, grain proteins such as oat, soy, wheat, buckwheat (average molecular mass, for example, 4,000-9,500 D), milk protein (2,500-8,000 D), or mixtures thereof (oat/soy; buckwheat/soy/oat; buckwheat/milk; oat/wheat/milk) can also be particularly suitable.

Also preferred are balneological preparations that have the following components:

75-88% vegetable oils, preferably selected from among soy oil, wheat germ oil, above all also in a mixture with C₁₂₋₂₂ triglycerides,

0.5 to 2.5% lecithin (soy egg lecithin); sphingosine; or mixtures thereof.

0.5 to 1.5% protein component, particularly selected from among wheat protein (hydrolysate), MW 11,000-19,000 D, oat protein (hydrolysate) (MW 11,500-20,000 D), barley protein (hydrolysate) (MW 11,000 to 21,000 D), milk protein (hydrolysate) (MW 9,000 to 15,000 D), quaternized milk protein (for example: 4,000 to 12,000 D), MW or mixtures of such proteins, such as wheat-milk; barley+oat; milk+oat;

0.5 to 8 wt.-% additives selected from among consistency regulators; conditioners, pigments, perfumes, preservatives; active substances, such as, for example, ether oils, plant extracts, vitamins.

In another embodiment, balneological premix preparations are made available, having the following components:

25 to 27 wt.-% lipids, selected from among vegetable lipids, C₁₂₋₂₂ fatty acid glycerides, C₂₋₆-alcohol C₁₂₋₂₂ fatty acid esters, C₈₋₂₂ fatty alcohols, or mixtures thereof, furthermore 57 to 65 wt.-% surfactants, selected from among ethoxylated and/or propoxylated C₈ to C₂₀ fatty alcohols or their sulfate salts, ethoxylated and/or propoxylated C₁₂ to C₂₂ fatty acids having an EO or PO degree of 1 to 5, in each instance, above all 1 to 4 (whereby EO derivatives are preferred), C₁₂ to C₂₂ fatty acid amides, anionic, if applicable ethoxylated (0-4 EO) C₈ to C₂₀ fatty alcohol sulfate salts, particularly sulfate monoisopropanolamines; or mixtures of such surfactants; 1 to 4 wt.-% of one or more phospholipids such as lecithins; sphingolipids or mixtures thereof; 0.05 to 3 wt.-% of one or more proteins or protein hydrolysates selected from among rice, bean, or also maize, millet, soy, wheat, oat protein, having an average molecular mass (in each instance) of 11,000 to 19,000 D, milk protein having an average molecular mss of 8,500 to 18,000 D, quaternized milk protein; or mixtures of such proteins/protein hydrolysates; as well as 1 to 10 wt.-% additives, selected from among active substances such as, for example: ether oils, plant extracts, vitamins, consistency regulators, conditioners, pigments, perfumes, preservatives, or mixtures thereof.

In such preparations, one or more ethoxylated C₁₀ to C₁₈ fatty alcohols, one or more C₁₂ to C₁₈ fatty acid amides, one or more C₁₀ to C₁₈ (if applicable, ethoxylated) fatty alcohol (ether) sulfate salts, particularly sulfate monoisopropanolamine; or mixtures of the said oxylated fatty alcohol(s), fatty acid amide(s), and the said fatty alcohol sulfate salt(s) are preferred as the surfactant.

These preferred concentrate preparations as described can also be used, above all, in the application forms processed with the galenic formulation aids as described, such as salts, tablets, shower creams or bath creams, bath foams, shower foams, bath beads, as described.

### Forms of Administration and Production of the Preparations

The preparation concentrates are produced in that the lipid or the lipids is/are mixed with the surfactant or the surfactants, by means of stirring, and subsequently, the liposome-forming substance is mixed together with (temperature-stable) additives that are present, if applicable, at elevated temperature, if applicable, for example up to 56° C., and subsequently, at room temperature the protein or the proteins as well as any temperature-stable additives that are present are worked in by means of stirring or the like.

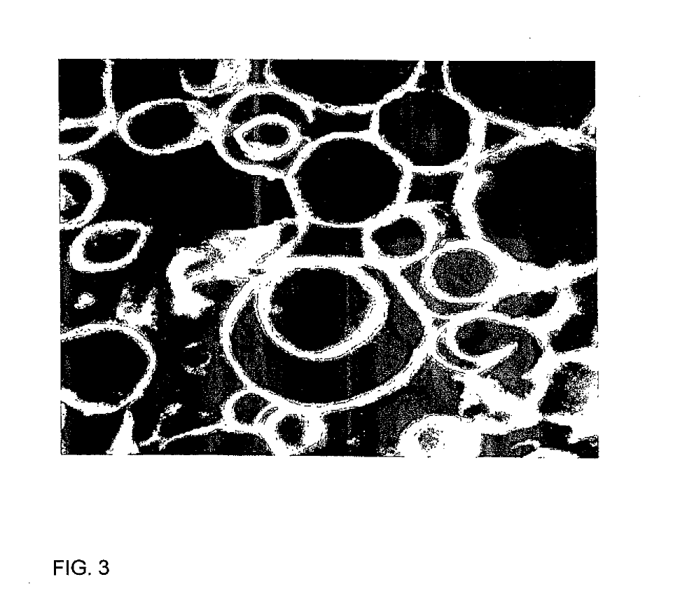

In particular, pourable to liquid products are present, which can be used as such, preferably in the tub: when they are poured into the warm water, proteoliposomes are spontaneously formed, as is evident from the attached TEM image according to FIG. 3.

Analogously, corresponding proteosomal effects are obtained if the concentrates are converted with galenic agents such as liquid solvents (particularly water) or solid agents such as salts, tablet-forming agents, colloids. The production of these products by means of mixing the concentrates with the galenic formulation agents as stated above, in suitable mixing and pressing devices, is known to a person skilled in the art and takes place in known manner.

### Use

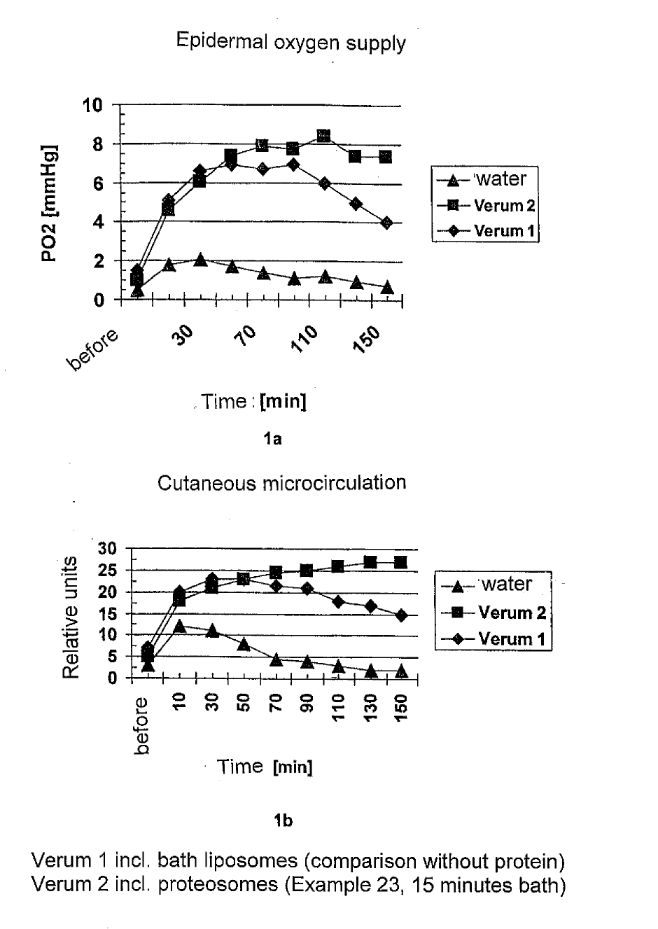

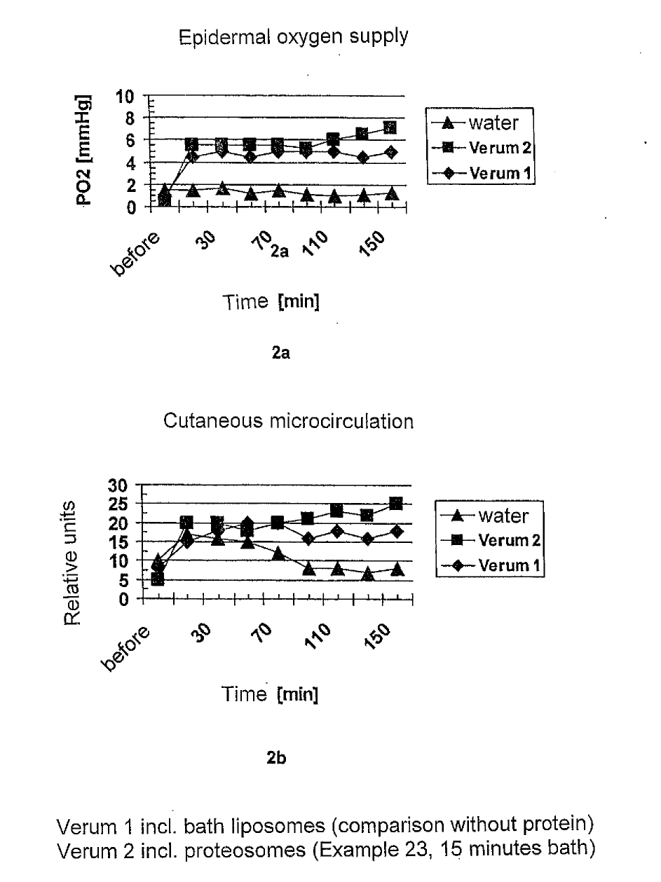

The preparations according to the invention are conceived, above all, for balneological-cosmetic use, for example as a relaxing, aroma-therapy, refreshing skin care bath (bath oil), foot bath, foam bath oil, or also as a shower bath and the like. In this connection, the corresponding active substances can be transported particularly effectively, as is also evident from FIGS. 1a, b and 2a, b.

They can also be formulated as indication baths, such as a rheumatic bath, revitalization bath, cold-treatment bath and relaxation bath, refreshing bath/shower. In particular, the concentrates or the solid preparations such as salts are suitable for this. For shower applications, preparations diluted with solvents can also be used.

### Exemplary Embodiments

The following balneological concentrates (Examples 1 to 4, Table 1) as well as galenic preparations made from them (Table 2 to 4, Examples 5-22) were produced as described above, and have the indicated amounts of substances (with reference to the total weight in wt.-%). The epidermally and cutaneously improved effectiveness or the formation of the proteosomal structure is evident from Examples 23-25.

TABLE 1
Liquid bath concentrates with proteosomes

| Ingredient | Example No. | 1 | 2 | 3 | 4 |
|---|---|---|---|---|---|
| Lipid | Jojoba oil | 15% | | | |
| | Soy oil | 30% | 55% | 15% | |
| | Almond oil | | 10% | | |
| | Paraffin oil | 30% | | | |
| | Isopropyl | | 10% | 12% | |
| | myristate | | | | |
| | Octyldecanol | 15% | | | |
| | Olive oil | | | | 10% |
| | Sunflower oil; | | | | 10% |
| | Glyceryl | | | | 27% |
| | monostearate | | | | |
| Protein | Wheat protein- | 0.5% | | | 0.3% |
| | hydrol. | (7500D) | | | (13000) |
| | Milk protein- | 0.5% | 0.5% | | |
| | hydrol. (3900D): | | | | |
| | Oat protein | | 0.3% | | |
| | (12000D) | | | | |
| | Milk protein hydr. | | | | 0.5% |
| | quater. (8000D) | | | | |
| | Barley protein | | | 1% | |
| | hydr. (12000D) | | | | |
| Liposome- | Sphingosine | 2% | | | 1% |
| forming | Soy lecithin | | 3% | | 2% |
| agent | Phosphatidyl | | | 2% | |
| | choline | | | | |
| Surfactant | Laureth2 | 2% | | | |
| | Laureth3 | | | | 35% |
| | Laureth4 | | 15% | 45% | |
| | MIPA-laureth | | | 9% | 10% |
| | sulfate (HLB 6) | | | | |
| | Cocoamide | | | 10% | |
| | diethanolamide | | | | |
| | PEG-400 | | | 5% | |
| Additives/ | Ivy leaf extract | 2% | | | |
| Active | Pine needle | | | | |
| substances | extract | | | | |
| | Vitamin E acet. | 1% | | 0.2% | 1.5% |
| | Rosemary oil | | 2.2% | | 1.5% |
| | Menthol | | 1% | | |
| | Eucalyptus oil | | 3% | | |
| | Lavender oil | | | | |
| | (eth.) | | | | |
| | Orange oil | | | | |
| | (eth.) | | | | |
| | Rosewood oil | | | 0.25% | |
| | Perfume oil | 2% | | 0.25% | 1% |
| | Pigment | | | 0.30% | 0.2% |

TABLE 2

| | | | Bath tablets | | Bath salt | | Pellets | |
|---|---|---|---|---|---|---|---|---|
| | | | containing | | containing | | containing | |
| Solid bath preparations | | | lipids | | lipids | | lipids | |
| With proteosomes | | | Prototype | Range | Prototype | Range | Prototype | Range |
| Examples 5-10 | | | (5) | (6) | (7) | (8) | (9) | (10) |
| Proteosome | Proteosome | Lipid | 3 | 0.5-9 | 3 | 0.5-9 | 3 | 0.5-9 |
| concentrate | premix | Protein | 2 | 0.3-7 | 1.5 | 0.3-7 | 2 | 0.3-7 |
| | | Liposome | 2 | 0.3-7 | 1.5 | 0.3-7 | 2 | 0.3-7 |
| | | Active | 3 | 0.5-9 | 2.5 | 0.4-8 | 2.5 | 0.4-8 |
| | | substance | | | | | | |
| | | Surfactant | 3 | 1-6 | 2 | 1-6 | 1.5 | 0.6-7 |
| | | Perfume, | 2 | | 1.5 | 1-9 | 2 | |
| | | pigment | | | | | | |
| Galenic | | NaCl or. | | | 45 | 33-68 | 83 | 78-95 |
| formulation | | pellets | | | | | | |
| agent | | CO₂-gen. | 44 | 35-51 | 21 | 15-39 | | |
| | | acidic | | | | | | |
| | | CO₂-gen. basic | 38 | 31-47 | 18 | 13-35 | | |
| | | Tablet. aid, | 3 | 1-6 | 4 | 1-8 | 4 | 1-8 |
| | | nutrient. | | | | | | |
| Total | | | 100 | | 100 | | 100 | |

TABLE 3

| | | | | | | | Foam bath | |
|---|---|---|---|---|---|---|---|---|
| Liquid bath | | | Emulsified | | Lipid-rich | | containing | |
| preparations | | | cream bath | | foam bath | | lipids | |
| with proteosomes | | | Prototype | Range | Prototype | Range | Prototype | Range |
| Examples 11-16 | | | (11) | (12) | (13) | (14) | (15) | (16) |
| Proteosome | Proteosome | Lipid | 29 | 14-45 | 52 | 25-67 | 18 | 5-25 |
| concentrate | premix | Protein | 3 | 0.2-11 | 3 | 0.2-11 | 3 | 0.2-11 |
| | | Liposome | 3 | 0.2-11 | 3 | 0.2-11 | 3 | 0.2-11 |
| | | Active | 4 | 0.4-14 | 4 | 0.4-14 | 4 | 0.4-14 |
| | | substance | | | | | | |
| | | Surfactant | 37 | 18-34 | 34 | 18-61 | 43 | 15-68 |
| | | Perfume, | 2 | | 1 | | 2 | |
| | | pigment | | | | | | |
| | | Preservative | 1 | | 2 | | 1 | |

| Water | 21 | 1 | 26 |
|---|---|---|---|
| Total | 100 | 100 | 100 |

TABLE 4

| | | | | | | | Shower foam | |
|---|---|---|---|---|---|---|---|---|
| Liquid shower | | | Emulsified | | Lipid-rich | | containing | |
| preparations | | | shower cream | | shower foam | | lipids | |
| with proteosomes | | | Prototype | Range | Prototype | Range | Prototype | Range |
| Examples 17-22 | | | (17) | (18) | (19) | (20) | (21) | (22) |
| Proteosome | Proteosome | Lipid | 14 | 8-35 | 43 | 25-62 | 8 | 3-25 |
| concentrate | premix | Protein | 2 | 0.2-8 | 2 | 0.2-8 | 2 | 0.2-8 |
| | | Liposome | 2 | 0.2-8 | 2 | 0.2-8 | 2 | 0.2-8 |
| | | Active | 3 | 0.4-11 | 3 | 0.4-11 | 3 | 0.4-11 |
| | | substance | | | | | | |
| | | Surfactant | 31 | 15-49 | 47 | 22-59 | 34 | 17-52 |
| | | Perfume, | 1.6 | | 1.5 | | 1.5 | |
| | | pigment | | | | | | |
| | | Preservative | 0.5 | | 0.5 | | 0.5 | |

| Water | 46 | 28-55 | 1 | 49 | 34-58 |
|---|---|---|---|---|---|
| Total | 100 | | 100 | 100 | |

### Example 23

A bath concentrate (Verum 2, Example 23) (skin-tightening bath concentrate) analogous to Example 12, comprising 25% water and 75% of the concentrate according to Example 1, with the following modifications* with regard to the lipids or replacement of the active substances and additives as follows: paraffin oil: 25%, soy oil: 24%, 5% rosemary oil, 6% pine needle oil, 1.5% ivy leaf extract, 1.5% niacin, 0.5% tocopheryl acetate, 1.5% methyl nicotinate was tested. According to the method according to E. Nürnberg, T. Gassenmeier, H. P. Albrecht, and O. P. Hornstein, Deutsche Apothekerzeitung [German Pharmacist Journal], 133^{rd} year, Number 41/1993, page 3715 to 3722, particularly page 3721, the cutaneous microcirculation (dermal perfusion) was determined by means of laser Doppler flowmetry (LDF), by way of the intravasal blood cell movement, by means of frequency shift of the laser light, by way of sensors affixed to the arms in appropriate manner. The epidermal oxygen supply was determined (by way of measuring the oxygen partial pressure), and the cutaneous microcirculation (erythrocyte flowmetry) was measured before and after before and after a 15-minute bath application (T=38° C., n=3), as described above or elsewhere, loc. cit., in comparison with water and with an analogous preparation (Verum 1), without protein. The results are shown in FIG. 1a/b. As is evident, a surprising improvement takes place with the preparation according to the application.

### Example 24

A preparation (skin-tightening shower concentrate) (Verum 2, Example 24) according to Example 18, with 46% water and 54% concentrate according to Example 4, with the following modifications* with regard to the lipids, proteins or replacement of the additive/active substances as indicated: 22% glycerol monostearate, 0.5% wheat protein hydrolysate as indicated; 30% laureth-3; 3% rosemary oil, 1% tocopheryl nicotinate, 1.5% ivy leaf extract, 3% pine needle oil, 1% niacin, 1.5% methyl nicotinate, 3% peeling body (without perfume/pigment) was tested. The cutaneous microcirculation (erythrocyte flowmetry) was determined before and after before and after a 5-minute shower application (T=38° C., n=3), as described above or elsewhere, loc. cit., in comparison with water and with an analogous preparation (Verum 1), without protein. Furthermore, the epidermal oxygen supply was as described above and elsewhere, loc. cit, by means of polarographic measurement of the oxygen partial pressure (platinum electrode) and determination of the measurement flow brought about as the result of the dermal O₂ diffusion by means of the O₂ reduction that took place at the electrode. The result (Verum 2), in each instance, is shown in FIG. 2, a, b, in comparison with water and the analogous preparation without protein (Verum 1). As is evident, an unexpected improvement takes place with the preparation Verum 2 according to the application, as compared with Verum 1.

* Modifications means: Change in the amount of the indicated components, while maintaining the other components (not listed again) of the substance group, in each instance—in contrast to replacement.

From these examples, the following is evident: The significant increase in the cutaneous microcirculation and the epidermal oxygen supply is proof of the intensive active substance transport by means of proteosomes, into deeper skin layers. In the case of balneological preparations that contain proteosomes, for “rinse-off” use, this is a particularly surprising increase in effect as compared with balneological preparations produced according to the state of the art.

### Example 25

A preparation according to Example 2 was introduced into bathwater (in an amount of more than 100 times as much water), and the structure that formed was determined in the electron microscope (TEM image). The image according to FIG. 3 shows the multi-layer structures that were surprisingly formed in this connection.

1. Balneological proteosome concentrates, comprising:
25 to 95 wt.-% lipids, selected from among vegetable lipids, C₁₂₋₂₂ fatty acid glycerides, C₂₋₆-alcohol C₁₂₋₂₂ fatty acid esters, C₈₋₂₂ fatty alcohols, paraffins, or mixtures thereof;
0.1 to 15 wt.-% of one or more liposome-forming substances, selected from among phospholipids, sphingolipids, or mixtures thereof;
0.5 to 70 wt.-% of one or more surfactants, selected from among polyoxyethylated and/or polyoxypropylated C₈-C₂₀ fatty alcohols or sulfate salts thereof; polyoxyethylated and/or polyoxypropylated C₁₂-C₂₂ fatty acids having an EO or PO or EO/PO degree of 1-5; C₁₂-C₂₂ fatty acid amides; mono sugar radicals of C₁₂-C₂₂ fatty acids or such polyoxyethylated and/or polyoxypropylated fatty acids with 2-16 EO, PO, or mixtures thereof, mono sugar ethers of C₈-C₂₂ fatty alcohols or ethoxylated and/or propoxylated fatty alcohols with 2-15 EO/PO/or EO-PO; or mixtures thereof;
0.5 to 15 wt.-% of one or more proteins, selected from among vegetable proteins having an average molecular mass of 4,000 D to 30,000 D, milk proteins having an average molecular mass of 2,500 to 18,000 D, quaternized milk proteins having an average molecular mass of 2,500-12,000 D, and, if applicable, additional additives, selected from among active substances, conditioners, consistency regulators, preservatives, wherein the amount of water amounts to less than 5 wt.-%.

2. Concentrate according to claim 1, further comprising 0.1 to 30 wt.-% additives, selected from among active substances, consistency regulators, conditioners, preservatives, pigments, perfumes, or mixtures thereof.

3. Preparations according to claim 1, wherein the surfactant(s) is/are selected from among nonionic ethoxylated and/or propoxylated C₈ to C₂₀ fatty alcohols, ethoxylated and/or propoxylated C₁₂ to C₂₂ fatty acids, having an EO or PO degree, in each instance, of 1-5, C₁₂ to C₂₂ fatty acid amides, particularly diethanolamides; anionic, if applicable ethoxylated C₈-C₂₀ fatty alcohol sulfate salts having an EO degree of 0-4; or mixtures thereof.

4. Preparation according to claim 1, wherein the preparation contains 75 to 90 wt.-% lipids, selected from among vegetable oils, C₁₂₋₂₂ fatty acid glycerides, C₂₋₆-alcohol C₁₂₋₂₂ fatty acid esters, C₈₋₂₂ fatty alcohols; caprylic-capric triglyceride, glyceryl stearate, or mixtures thereof, furthermore 5 to 15 wt.-% surfactants, selected from among ethoxylated and/or propoxylated C₈ to C₂₀ fatty alcohols, ethoxylated and/or propoxylated C₁₂ to C₂₀ fatty acids, having an EO or PO degree of 1 to 5, in each instance, C₁₂ to C₂₀ fatty acid amides, or mixtures thereof; 2 to 4 wt.-% of one or more lecithins, sphingolipids, or mixtures thereof; 0.5 to 4 wt.-% of one or more proteins or protein hydrolysates, selected from among rice, bean, or also maize, millet, soy, wheat, oat protein having an average molecular mass of 4,000 D to 25,000 D, in each instance, milk protein having an average molecular mass of 2,500 to 18,000 D, or quaternized milk protein having an average molecular mass of 2,500 D to 12,000 D, as well as 1 to 10 wt.-% additives, selected from among active substances, consistency regulators, conditioners, preservatives, pigments, perfumes, or mixtures thereof.

5. Preparation according to claim 1, wherein it comprises 25 to 27 wt.-% lipids, selected from among vegetable lipids, C₁₂₋₂₂ fatty acid glycerides, C₂₋₆-alcohol C₁₂₋₂₂ fatty acid esters, C₈₋₂₂ fatty alcohols, or mixtures thereof; furthermore 57 to 65 wt.-% surfactants, selected from among ethoxylated and/or propoxylated C₈ to C₂₀ fatty alcohols or their sulfate salts, ethoxylated and/or propoxylated C₁₂ to C₂₀ fatty acids having an EO or PO degree of 1 to 5, in each instance, above all 1 to 4 (whereby EO derivatives are preferred), C₁₂ to C₂₀ fatty acid amides, anionic, if applicable ethoxylated (0-4 EO) fatty alcohol (ether) sulfate salts, particularly sulfate monoisopropanolamines; or mixtures of such surfactants; 1 to 4 wt.-% of one or more phospholipids, sphingolipids or mixtures thereof; 0.05 to 3 wt.-% of one or more proteins or protein hydrolysates selected from among rice, bean, or also maize, millet, soy, wheat, oat protein, having an average molecular mass (in each instance) of 4,000 to 19,000 D, milk protein having an average molecular mass of 2,500 to 18,000 D, quaternized milk protein having an average molecular mass of 2,500 to 12,000 D, or mixtures of such proteins/protein hydrolysates; as well as 1 to 10 wt.-% additives, selected from among active substances, consistency regulators, conditioners, pigments, perfumes, preservatives, or mixtures thereof.

6. Preparation according to claim 1, wherein in it, the surfactant that is present is at least one surfactant selected from the group comprising ethoxylated and/or propoxylated C₁₀ to C₂₀ fatty alcohols, ethoxylated and/or propoxylated C₁₂ to C₂₀ fatty acids having an EO or PO or EO-PO degree of 1-5, C₁₀ to C₂₀ (if applicable, ethoxylated) fatty alcohol (ether) sulfate salts, sulfate monoisopropanolamine having an EO degree of 0.4; or mixtures thereof.

7. Preparation according to claim 1, wherein one or more ethoxylated C₁₀ to C₁₈ fatty alcohols, one or more C₁₂ to C₁₈ fatty acid amides, one or more C₁₀ to C₁₈ (if applicable, ethoxylated) fatty alcohol (ether) sulfate salts, particularly sulfate monoisopropanolamine; or mixtures of the said oxylated fatty alcohol(s), fatty acid amide(s), and the said fatty alcohol sulfate salt(s) are present as the surfactant.

8. Preparation according to claim 1, wherein as protein(s), rice, bean or also maize, millet, soy, wheat, oat protein or hydrolysates having an average molecular mass of 11,000 D-19,000 D, or mixtures thereof, or milk proteins having an average molecular mass of 8,500 to 15,000 D or mixtures of the said milk protein with the same one or more grain protein(s) are present.

9. Preparations according to claim 1, wherein a vegetable protein, particularly grain protein, having an average molecular mass of 11,000 D to 25,000 D, in a mixture with quaternized milk protein having an average molecular mass of 2,500 D to 12,000 D is contained in a total amount of 0.5 to 3 wt.-%.

10. Preparation according to claim 1, wherein as protein(s), one or more vegetable proteins selected from among rice, bean, maize, millet, soy, wheat, oat protein or protein hydrolysates, having an average molecular mass of 4,000 D to 9,500 D, or milk protein or quaternized milk protein, having an average molecular mass of 2,500 D to 3,500 D, or 8,500 D to 15,000 D, or mixtures of the said milk protein with one or more said grain proteins, are present.

11. Preparation according to claim 1, wherein the weight ratio of liposome-forming substance to protein amounts to 1:1 to 5:1.

12. Preparation according to claim 1, wherein vegetable oils or vegetable oils in a mixture with C₁₂₋₂₂ fatty acid glycerides and/or C₈₋₂₂ fatty alcohols are contained.

13. Preparation according to claim 12, wherein the vegetable lipid or the vegetable lipids is/are selected from among jojoba oil, sunflower oil, almond oil, olive oil, or mixtures thereof.

14. Galenic preparation in the form of a liquid or solid bath or shower additive, wherein in it are contained 5-84% of a concentrate according to claims 1 and 95 to 16 wt.-% of one or more galenic aids, selected from among solid regenerative substances, tablet-forming substances, solid gas/foam generators, salt crystals, organic pellets, or liquid solvents, particularly water.

15. Galenic preparation according to claim 14, selected from among:
a) products comprising 82 to 95 wt.-% solid salt media, if applicable 0 to 8 wt.-% tablet-forming aid, 5 to 28 wt.-% premix concentrate according to claim 1;
b) products comprising 90 to 95 wt.-% colloid and 5 to 10 wt.-% premix concentrate according to claim 1;
c) products comprising 40 to 58 wt.-% water and 42 to 60 wt.-% premix concentrate according to claim 1;
d) products comprising 16 to 34 wt.-% water and 66 to 84 wt.-% premix concentrate according to claim 1.

16. Use of a concentrate according to claim 1 or of a balneological preparation containing same as a bath oil, foam bath oil, bath cream, foam bath cream, shower cream, shower bath cream, bath salt, bath pellets, foot bath, indication bath such as relaxation bath, conditioning bath, aroma-therapy bath, refreshing bath.

17. Use of a concentrate according to claim 1 for the production of a product for full/partial shower baths, foot baths, bath salts or bath powders, bath pellets.

18. Use of a concentrate or of a preparation according to claim 1 for the production of a product for indication or healing baths, such as cold-treatment baths, rheumatic baths, revitalization baths.
